(19) United States
(12) Patent Application Publication
(10) Pub. No.: US 2007/0148090 A1
(43) Pub. Date: Jun. 28, 2007

(54) Per (3,6-anhydro) cyclodextrin derivatives, preparation thereof and use thereof for transporting metal elements to biological targets or for decontaminating biological targets of fluids

(76) Inventors: Cecile Baudin, Paris (FR); Bruno Perly, St. Denis (FR); Jean-Pierre Dalbiez, Gif-Sur-Yvette (FR)

Correspondence Address: HUTCHISON LAW GROUP PLLC, PO BOX 31686, RALEIGH, NC, 27612, US

(21) Appl. No.: 10/544,680

(22) PCT Filed: Feb. 6, 2004
(86) PCT No.: PCT/FR04/50048

§ 371 Date: May 15, 2006

(30) Foreign Application Priority Data
Feb. 7, 2003 (FR) 03/01474

(51) Int. Cl. A61K 51/00; A61K 38/14

(52) U.S. Cl. 424/1.69; 514/58; 514/8; 530/322

(57) ABSTRACT

The present invention relates to a per(3,6-anhydro)cyclodextrin derivative corresponding to one of the following formulae (I) or (II): 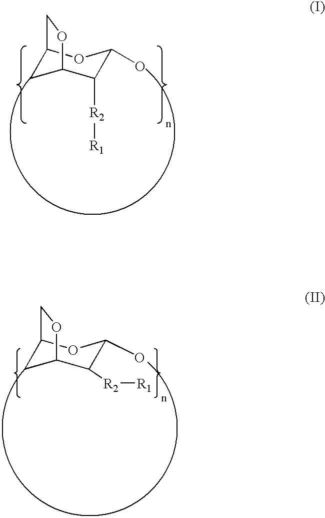
in which: at least one of the values R¹ represents a radical chosen from peptides, proteins, lipids, oligonucleotides or polynucleotides, oligosaccharides or polysaccharides, and biopolymers, and the other possible values R¹, which may be identical or different, represent a group chosen from OH, OR³, OM, SH, SR³, OCOR³, NH₂, NHR²,NR³R⁴, CONH₂, CONHR³, CONR³R⁴, CN, COOR³, OCH₂COOH, COOH, OSO₂R³, N₃ and R³, in which R³ and R⁴, which may be identical or different, represent a saturated or unsaturated, aliphatic or aromatic, hydrocarbon-based group optionally substituted with halogen atoms, which may contain one or more hetero atoms chosen from O, S and N, M represents a monovalent cation chosen from alkali metal cations; the values R² represent a single bond or a spacer group, and n is equal to 6, 7 or 8. These derivatives are used in particular for transporting metal elements to biological targets or for decontaminating biological targets or fluids.

### TECHNICAL FIELD

The present invention relates to novel chemically modified per(3,6-anhydro)cyclodextrin derivatives that can be used for attaching metal elements, and more particularly for transporting, due to the chemical modification, such elements to a biological target or eliminating these elements from a biological target or a biological fluid.

The present invention also relates to crosslinked per(3,6-anhydro)cyclodextrins, it being possible for said per(3,6-anhydro)cyclodextrin derivatives to be used for eliminating metal elements from a biological fluid.

The present invention can be applied in particular in the diagnostic field, in the field of therapy with metal elements, or else in the field of human decontamination in vitro and in vivo.

### Prior State of the Art

Cyclodextrins, of cyclomaltooligosaccharides, are compounds of natural origin formed by the cyclic linking of α-(1,4)-linked glucose units. Derivatives thereof may consist of α-(1,4)-linked maltose units.

Many studies have shown that these compounds can form inclusion complexes with hydrophobic molecules, thus allowing them to be solubilized in aqueous media. Many applications have been proposed in order to take advantage of this phenomenon, in particular in the pharmaceutical field, as is described by D. Duchêne, “Pharmaceutical application of cyclodextrins” in “Cyclodextrins and their industrial uses”, D. Duchêne Ed., Editions de Santê, Paris, 1987, pages 213-257 [1].

Among the large number of modified derivatives of these cyclodextrins, those for which the cavity is turned inside out exhibit advantageous properties, even if their ability to include organic molecules is lost or very limited. Compounds of this type are per(3,6-anhydro)cyclodextrins.

The synthesis of these per(3,6-anhydro)cyclodextrins was described as early as in 1991 in document [2]: Gadelle A. and Defaye J., Angew. Chem. Int. Ed. Engl., (1991), 30, pages.78-79; and document [3]: Ashton P. R., Ellwood P., Staton I. and Stoddart J. F., Angew. Chem. Int. ed. Engl., (1991) 30, pages 80-81, and it has been shown that these derivatives exhibit advantageous solubilities both in water and in organic solvents. Some subsequent studies (document [4] : Yamamura H. and Fujita K. Chem. Pharm. Bull., (1991) 39, pages 2505-2508; document [5]: Yamamura H., Ezuka T., Kawase Y., Kawai M., Butsugan Y. and Fujita K., J. Chem. Soc., Chem. Com., (1993) pages 636-637; and document [6]: Yamamura H., Nagaoka H., Kawai M. and Butsugan Y., Tetrahedron Lett. (1995) 36, pages 1093-1094) have also shown that these peranhydro derivatives can complex alkali metal ions with not insignificant selectivity. Document FR-A 2 744 124 [7], document FR-A 2 764 525 [8] and document FR-A 2 807 044 [9] mention other per(3,6-anhydro)cyclodextrin derivatives substituted in the 2-position, that are used for separating various ions, in particular potassium and caesium in the case of document [7], due to the presence of the acetyl substituent, or lead in the case of document [8], due to the presence of a methoxy substituent, or polluting ions such as the cobalt or uranyl ion and the lanthanide ions in the case of document [9], due to the presence of a substituent —O—CH₂—CO₂H.

However, these cyclodextrins are not peranhydrocyclo-dextrins described as transporters of metal elements to biological targets or which can be used, after crosslinking, for purifying a biological fluid contaminated with one or more toxic metal elements.

### DISCLOSURE OF THE INVENTION

A subject of the present invention is novel peranhydrocyclodextrin derivatives in which the substituent in the 2-position makes it possible to transport said derivatives to a biological target, while conferring metal element-complexing, for example radioactive element-complexing, properties for the purpose of the possible use of these derivatives in the radiodiagnostic field, the radiotherapy field or the human decontamination field.

A subject of the present invention is also crosslinked per(3,6-anhydro)cyclodextrin derivatives, which crosslinking confers on these novel subjects a characteristic of insolubility in a biological fluid and makes it possible, due to the complexing properties of the per(3,6-anhydro)cyclodextrins, to purify said biological fluid in terms of toxic metal elements, in the context, for example, of a dialysis.

According to the invention, the per(3,6-anhydro)cyclodextrin derivative corresponds to one of the following formulae (I) or (II): 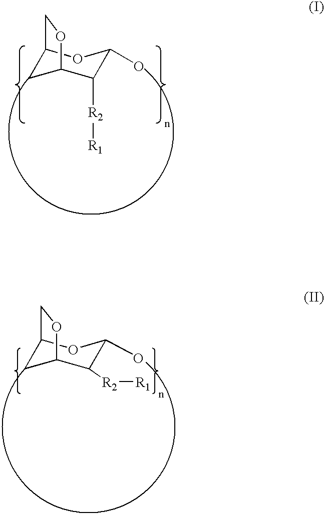
in which:
at least one of the values R¹ represents a radical chosen from peptides, proteins, lipids, oligonucleotides or polynucleotides, oligosaccharides or polysaccharides, and biopolymers, and the other possible values R¹, that do not correspond to this definition, represent groups, which may be identical or different, chosen from OH, OR³, OM, SH, SR³, OCOR³, NH₂, NHR³, NR³R⁴, CONH₂, CONHR³, CONR³R⁴, CN, COOR³, OCH₂COOH, COOH, OSO₂R³, N₃ and R³, in which R³ and R⁴, which may be identical or different, represent a saturated or unsaturated, aliphatic or aromatic, hydrocarbon-based group optionally substituted with halogen atoms, which may contain one or more hetero atoms chosen from O, S and N, and M represents a monovalent cation chosen from alkali metal cations;
the values R², which may be identical or different, represent a single bond or a spacer group;
n is equal to 6, 7 or 8.

Thus, the derivatives of the invention comprise at least one of the groups R¹ representing a radical chosen from peptides, proteins, lipids, oligonucleotides or polynucleotides, oligosaccharides or polysaccharides, and biopolymers, and, optionally (when not all the values R¹ correspond to this definition), one or more groups R¹ representing groups, which may be identical or different, chosen from OH, OR³, OM, SH, SR³, OCOR³, NH₂, NHR³, NR³R⁴, CONH₂, CONHR³, CONR³R⁴, CN, COOR³, OCH₂COOH, COOH, OSO₂R³, N₃ and R³.

In the cyclodextrin derivative of formula (I) or (II)., the aliphatic or aromatic hydrocarbon-based groups that can be used for R³ and R⁴ may be of various types. They may consist of a carbon chain in which some carbon atoms can be replaced with one or more hetero atoms such as O, S and N, and they may contain one or more ethylenic or acetylenic unsaturations. Moreover, the hydrocarbon-based group may be substituted with halogen atoms. The aromatic hydrocarbon-based groups that can be used for R³ and R⁴ may consist of the phenyl group and the tosyl group, optionally substituted, for example, with alkyl groups containing from 1 to 20 carbon atoms.

The aliphatic hydrocarbon-based groups that can be used to constitute the values R³ and R⁴ may, in particular, represent a linear or branched alkyl group containing from 1 to 20 carbon atoms.

R² may represent a single bond, in particular when the values R¹ are groups chosen from OH, OR³, OM, SH, SR³, OCOR³, NH₂, NHR³, NR³R⁴, CONH₂, CONHR³, CONR³R⁴, CN, COOR³, OCH₂COOH, COOH, OSO₂R³, N₃ and R³. In this case, the groups R¹ are directly covalently attached to the cyclic carbon of the glucose or maltose units.

R² may represent a spacer group, i.e. an organic-type group which is not part of the radicals of peptide, protein, lipid, oligonucleotide or polynucleotide, and oligosaccharide or polysaccharide nature. This spacer group may be a group chosen, for example, from —NH—, —NR—, —NH—(CH₂)m-CO₂—, —NH-—CH₂)m-NH—, —NH—CO₂—, —NH—CO—, —NH—CO—(CH₂)m-COO—, —NH—CO—(CH₂)m-CO—NH—, —NH—CO—(CH₂)m-NH—CO—, —NH—CO—CHR—NH—, —O—, —O—(CH₂)ₘ—, —O—(CH₂)ₘ—O—, —O—(CH₂)m-CO—, —O—(CH₂)m-CO₂—, —O—(CH₂)m-NH—, —O—(CH₂)m-NH—CO—, —O—(CH₂)m-CO——NH—, —O—CO—NH—, —O—CO—NR—, —O—CO—, —O—CO₂—, —S—, —S—(CH₂)m-CO—, —S—(CH₂)m-CO₂—, —S—(CH₂)m-NH—, —S—(CH₂)m-NH—CO—, —S—(CH₂)m-CO—NH—, —S—CO—NH—, —S—CO—, —S—CO—S—, —O—CS—S—, —CS—NR—, —O—CS₂—, —CO—, —CO—NH—, —CO—NR—, —CS—NR—, —CO—O—, —CO—S—, —CO—O—(CH₂)m-CO—O—, —CO—S—(CH₂)m-CO—O—, —CO—S—(CH₂)m-CO—S—, —CHOH—NH—, —CHSH—NH—, —CHOH—(CH₂)m-NH—, —CHSH—(CH₂)m-NH— with R having the same definition as R³ or R⁴; m is an integer ranging from 1 to 12.

In accordance with the definition of the peranhydrocyclodextrin derivatives of formula (I) or (II) defined above, at least one of the values R¹ is a radical chosen from peptides, proteins, lipids, oligonucleotides or polynucleotides, oligosaccharides or polysaccharides, and biopolymers.

According to the present invention, the term “peptide” is intended to mean a molecule consisting of a chain of 2 to 30 amino acids, it being possible for said amino acids to be identical or different, the linkage between two consecutive amino acids of the chain being effected by means of a loss of water between the amine group of one amino acid and the carboxyl group of the neighbouring amino acid, so as to form a —CO—NH— linkage.

The term “protein” is intended to mean a molecule formed by the linking together of a large number of amino acids, for example from 30 to 30 000.

The term “lipid” is intended to mean a molecule consisting of long-chain fatty acid esters, of long-chain fatty acid amides, of linear- or branched-chain saturated or unsaturated fatty acids, of aliphatic higher alcohols, of sterols (such as cholesterol), of hydrocarbons such as squalene, of liposoluble vitamins, of acylglycerols such as 1,2,3-triacylglycerol, of glycoglycerides, or of phospholipids.

The term “oligonucleotide or polynucleotide” is intended to mean a molecule consisting of a sequence of nucleotides, the number of which ranges from one to a few tens but is less than 100 for the oligonucleotides, such as oligo[A] (oligo(5′-adenylic acid)), oligo[I] (oligo(5′-inosinic acid)) or oligo[U] (oligo(5′-uridilic acid)), and greater than 100 for the polynucleotides such as poly[A] (poly(5′-adenylic acid)), poly[I] (poly(5′-inosinic acid)) or poly[U]. (poly(5′-uridilic acid)).

The term “oligosaccharide or polysaccharide” is intended to mean a molecule consisting of a chain of simple saccharide units, which may be identical or different (from 2 to 10 for the oligosaccharides, such as glucose or mannose, and more than 10 for the polysaccharides).

The term “biopolymer” is intended to mean a linear or branched chain of monomers, which may be identical or different, of biological origin (peptides, lipids, proteins, nucleotides, saccharides), such as poly-L-glutamic acid, poly-L-naphthylalanine, poly-L-phenylalanine, poly-L-tryptophan, poly-L-tyrosine, poly-L-histidine or poly-L-lysine.

According to a particular embodiment of the invention, the peranhydrocyclodextrin derivative used that corresponds to formula (I) is a derivative in which at least one of the values R¹ represents a radical of peptide nature, the other possible values R¹ representing —OH, the R² linked to R¹ that represents a radical of peptide nature represents a spacer group of formula —OCH₂CO—, the possible values R² linked to the possible values R¹ that represent —OH are single bonds, and n is equal to 6.

In particular, the radical of peptide nature may correspond to the formula —NH—CH(CH₃)—CO—NH—CHBz-CO—OCH₃, Bz corresponding to a benzyl group, i.e. a group of formula:

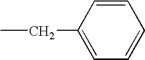

According to another particular embodiment of the invention, the peranhydrocyclodextrin derivative corresponding to formula (I) is a derivative in which at least one of the values R¹ represents a biopolymer, the other possible values R¹ representing —OH, the R² linked to R¹ that represents a biopolymer represents a spacer group of formula —OCH₂CO—, the possible values R² linked to the possible values R¹ that represent —OH are single bonds, and n is equal to 6.

In particular, the biopolymer may correspond to a poly-L-lysine. This poly-L-lysine may have a molecular weight ranging from 3000 to 300 000, for example a molecular weight equal to 7500.

The derivatives in accordance with the present invention may, in particular, have an ability to complex metal elements that is greater than that of the basic cyclodextrin from which they are derived, as will be illustrated more fully in the experimental section of this description.

In addition, because of the presence of such radicals, they exhibit the following advantages:
they are capable of being transported to a given biological target; as a result, they can be used for transporting metal elements to a given biological target, for the purpose of treating this target with said metal element;
they can be used for decontaminating, with respect to a metal element, a biological target, it being understood that the choice of the radical derived from a biomolecule will be made according to the biological target to be decontaminated and according to the metal element to be eliminated;
they are biocompatible, i.e. well tolerated by a live organism, and can therefore be used, for example, for decontaminating a biological fluid;
they are capable of being crosslinked, at the level of the radicals when the latter are biopolymers, thus being able to form polymers that are insoluble in a given biological liquid; as a result, they can be used for decontaminating a biological liquid in vitro.

Thus, the derivatives of the present invention can also be crosslinked, as is emphasized in the above paragraph.

It is specified that, according to the invention, the term “crosslinked derivative” is intended to a mean a derivative obtained by crosslinking a derivative comprising at least one R¹ of biopolymer type, said crosslinking being carried out by crosslinking said biopolymer. This crosslinking can be envisaged according to several embodiments.

According to a first embodiment, the crosslinked derivative of the invention results from the intermolecular crosslinking of at least two different per(3,6-anhydro)cyclodextrins, said two per(3,6-anhydro)cyclodextrins being linked by means of a unit forming a bridge between two values R¹ representing a radical of biopolymer type.

According to a second embodiment, the crosslinked derivative according to the invention may result from intramolecular crosslinking between at least two values R¹ of a single base per(3,6-anhydro)cyclodextrin, said values R¹ in question being radicals of biopolymer type. The resulting crosslinked derivative is in the form of a per(3,6-anhydro)cyclodextrin derivative in which at least two values R¹ of biopolymer type are connected by a bridge-forming unit.

It is also possible to envisage, according to the invention, the case of crosslinked derivatives resulting from the combination of the abovementioned two embodiments, namely a crosslinked derivative resulting both from an intermolecular crosslinking and from an intramolecular crosslinking.

It is understood that, according to the invention, the values R¹ of biopolymer type that are involved in the intermolecular and/or intramolecular crosslinking contain, before crosslinking, reactive functions, so that the crosslinking can take place by the simple action of a crosslinking agent comprising at least two reactive functions capable of reacting with said reactive functions carried by the abovementioned values R¹.

A particular crosslinked derivative of the invention is a derivative for which the per(3,6-anhydro)cyclodextrin derivative acting as a basis for the crosslinking is a derivative for which all the values R¹ are radicals of poly-L-lysine type and the values R² are spacer groups of formula —O—CH₂—CO—, n being equal to 6, at least one of the abovementioned values R¹ being crosslinked by the action of glutaraldehyde in the presence of sodium borohydride.

In this particular case, the glutaraldehyde reacts with the primary amine functions carried by the poly-L-lysine, thus forming imine units 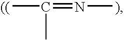
which are then reduced by the sodium borohydride so as to give units of formula 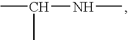
said units forming a bridge between two values R¹.

The crosslinked derivatives according to the invention, because of the presence of values R¹ derived from biopolymers and also of radicals derived from values R¹ partially converted by crosslinking, exhibit a good capacity for complexation with metal elements. In addition, because of the crosslinking, they can constitute compounds that are insoluble in a given physiological liquid. In this case, these polymers find an application in the decontamination of biological fluids with respect to toxic metal elements. They can in particular be adsorbed onto membranes through which the biological fluid to be decontaminated circulates. These derivatives may therefore be particularly advantageous in the context of dialysis treatments of contaminated biological fluids.

The derivatives of the invention corresponding to formula (I) or (II) given above, in which at least one of the values R¹represents a radical as defined above, the other values R¹ representing OH or another group as mentioned above and n being equal to 6, 7 or 8, can be prepared by means of a method comprising the following steps:
1) reacting a peranhydrocyclodextrin corresponding to the following formulae (III) or (IV): 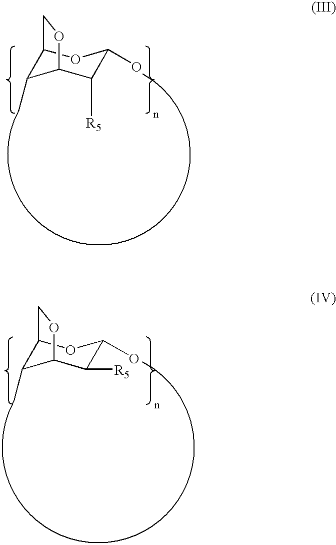
   in which the values R⁵, which may be identical or different, represent an OH group or a group that is a precursor of the spacer group R², with a biomolecule chosen from peptides, proteins, lipids, oligonucleotides or polynucleotides, oligosaccharides or polysaccharides, and biopolymers, said biomolecule comprising a reactive group capable of reacting with the group R⁵ as defined above;
2) optionally, reacting the values R⁵ that have not reacted with the abovementioned biomolecule, with one or more suitable reactants so as to convert them to the desired groups R¹.

According to the invention, said group that is a precursor of the spacer group R² may be a group chosen from —NH₂, —NHR, —NH—(CH₂)m-CO₂H, —NH—(CH₂)m-NH₂, —NH—CO₂H, —NH—COX, —NH—CO—(CH₂)m-COOH, —NH—CO—(CH₂)m-CO—NH₂, —NH—CO—(CH₂)ₘ—NH—COX, —NH—CO—CHR—NH₂, —OH, —O—(CH₂)m-X, —O—(CH₂)m-COX, —O—(CH₂)m-CO₂H, —O—(CH₂)m-NH₂, —O—(CH₂)m-NH—COX, —O—(CH₂)m-CO-NH₂, —O—CO—NH₂, —O—CO—NHR, —O—COX, —O—CO₂H, —SH, —S—(CH₂) m-COX, —S—(CH₂)m-CO₂H, —S—(CH₂)m-NH₂, —S—(CH₂)m-NH—COX, —S—(CH₂)m-CO—NH₂, —S—CO—NH₂, —S—COX, —S—CO—SH, —O—CS—SH, —CS—NHR, —O—CS₂H, —CO₂H, —COSH, —CO—NHR, —CS—NHR, —CO—O—(CH₂)m-COOH, —CO—S—(CH₂)m-COOH, —CO—S—(CH₂)m-COSH, —CHOH—NH₂, —CHSH-NH₂, —CHOH—(CH₂)m-NH₂, —CHSH—(CH₂)m-NH₂, —O—(CH₂)m-OH, —COX and —CONH₂, with m having the same definition as that given above and X being a halogen atom.

In order to carry out the first step, the necessary amount of biomolecules of peptide, protein, lipid, oligonucleotide or polynucleotide, or oligosaccharide or polysaccharide nature or the biopolymer, to suitably modify at least one group R⁵ of the starting cyclodextrin, is used.

When the cyclodextrin derivative corresponds to formula (I) or (II) in which the other values R¹ represent OH and R² is a single bond, step 2) of the method is not to be carried out when all the values R⁵ of the starting cyclodextrin represent OH.

When the cyclodextrin derivative corresponds to formula (I) or (II) given above in which the other values R¹ represent OR³ with R³ having the meaning given above, the peranhydrocyclodextrin that has been partially modified, during the first step of the method, can be reacted with an alkali metal hydride in order to convert the —OH functions to —OM functions with M representing an alkali metal, and the derivative obtained is then reacted with a halide of formula R³X in which R³ has the same meaning given above and X is a halogen atom.

When the cyclodextrin derivative corresponds to formula (I) or (II) given above in which the other values R¹ represent —OCOR³, the process is initially carried out as above, and then the derivative obtained is subsequently reacted with an alkyl halide of formula R³ COX in which R³ has the meaning given above and X represents a halogen. The —OH functions can also be converted to —OCOR³ functions by reacting the cyclodextrin derivative directly with the acid anhydride of formula (R³CO) ₂O.

When it is desired to prepare a cyclodextrin derivative in which the other value(s) R¹ represent(s) a group of formula SH, SR³, NH₂, NHR³ NR³R⁴, CONR³R⁴, CONHR³, CONH₂, CN, COOR³, COOH, or R³, with R³ and R⁴ having the meanings give above, and n is equal to 6, 7 or 8, the following steps can be carried out, starting from a partially modified peranhydrocyclodextrin, i.e. one in which at least one of the values R¹ represents a radical of peptide, protein., lipid, oligonucleotide or polynucleotide, or oligosaccharide or polysaccharide nature, or a biopolymer:
1) reacting this peranhydrocyclodextrin with an alkali metal hydride so as to convert the OH group(s) to OM group(s) with M representing an alkali metal;
2) reacting the modified peranhydrocyclodextrin obtained in 1) with a chloride of formula ClSO₂R³ with R³ having the meaning given above, so as to obtain the derivative of formula (I) or (II) in which at least one of the values R¹ is a group of formula OSO₂R³; and
3) reacting the derivative obtained in the second step with one or more suitable reactants so as to replace OSO₂R³ with the desired group R¹.

In this method, the partially modified per(3,6-anhydro)cyclodextrin is first of all converted to an alkoxide by the action of an alkali metal hydride, and then this alkoxide is converted to a derivative comprising a leaving group of formula OSO₂R³, which is then reacted in one or more steps with one or more suitable reactants so as to replace this leaving group with the desired group R¹.

Thus, when R¹ should represent NH₂, N₃M and the compound defined in 2) can be reacted. The compound thus obtained, called azide, can be subjected to a catalytic hydrogenation or be treated in the presence of ammonia NH₃, in order to obtain the product in which R¹ should represent NH₂.

The product in which R¹ should represent NHR³ or NR³R⁴ is obtained by reacting the compound defined in 2) with the compound NH₂R³ or NHR³R⁴.

When R¹ should represent SH or SR³, the compound defined in 2) can be reacted with a halide X⁻, which gives the compound with (R¹=X), which is then reacted with HS⁻ or R³S⁻, to give a compound in which R¹ represents SH or SR³.

When R¹ should represent a hydrocarbon-based group, the compound obtained in 2) is reacted with R³₂LiCu to give a final compound in which R³ then represents a hydrocarbon-based group, as defined above.

Similarly, the compound in which R¹ represents a halogen can react with CN⁻ to give a final compound in which R¹ will represent CN.

Similarly, the compound in which R¹ represents CN can, by controlled hydrolysis, give a compound in which R¹ will represent CONH₂. The compound in which R¹ represents CN can, by complete hydrolysis, give a compound in which R¹ will represent COOH.

The compound in which R¹ represents COOH can, by esterification, give a compound in which R¹ will represent COOR³.

The compound in which R¹ represents COOH can react with NHR³R⁴ or NH₂R³ in the presence of DCC (dicyclohexylcarbodiimide) to give a compound in which R¹ will represent NR³R⁴ or NHR³.

The grafting of the radicals of peptide, protein, lipid, oligonucleotide or polynucleotide, or oligosaccharide or polysaccharide nature, or of the biopolymers, onto a peranhydrocyclodextrin by reaction with the values R⁵ of the cyclodextrin of formula (III) or (IV) can be carried out in various ways.

For example, to graft a radical of peptide nature having a reactive —NH₂ end, this involves creating a peptide bond between the per(3,6-anhydro)cyclodextrin (3,6-CD) and the peptide. For this, the 3,6-CD may preferably have a group that ends with an —NH₂ or —COOH function.

When the acid end of a peptide is reacted with the cyclodextrin (in this case, with an —NH₂ function or derivative), the acid end must be converted to an acid halide, mixed anhydride, azide or ester that is activated. Once this end has been activated, it can react directly with the amine function of the cyclodextrin so as to form the peptide bond. The acid function can also be reacted with a coupling reagent such as dicyclohexylcarbodiimide.

The reaction consisting in grafting a radical of protein nature can be carried out in a similar manner to that disclosed above.

To graft a radical of lipid nature, this radical (when it corresponds in particular to a fatty acid) can be grafted onto an —NH₂ function, the coupling methods being similar to those disclosed for the coupling of a radical of peptide or protein nature.

To graft a radical of oligonucleotide or polynucleotide nature, use may be made of a DNA sequence containing an amine function on an end phosphate group. The coupling methods are, in this case, similar to those disclosed above.

To graft a radical of saccharide nature, the sugar can, for example, be functionalized in order to introduce therein a group capable of reacting with the R₅ of the cyclodextrin. This group capable of reacting may, for example, be an ethylenediamine group.

According to the invention, when at least one of the values R¹ is a radical of biopolymer type, the method may also comprise, after the grafting step and the optional step for converting the values R⁵ to suitable values R¹ (or to suitable groups R²—R¹, when R² is other than a single bond), a step consisting in crosslinking, by the action of a crosslinking agent such as glutaraldehyde, on the per(3,6-anhydro)cyclodextrin derivative previously converted.

As stated above, the per(3,6-anhydro)cyclodextrin derivatives in accordance with the invention have excellent capacities for complexation of metal elements due to the presence of the cavity of the per(3,6-anhydro)cyclodextrin and of the radicals carried by the per(3,6-anhydro)cyclodextrins in the 2-position.

Thus, a subject of the present invention is also complexes of a metal element with a peranhydrocyclo-dextrin derivative, as defined above.

In particular, the metal element forming a complex with a peranhydrocyclodextrin derivative, in accordance with the invention, can be chosen from Tc, Y, In, Ga, Re, Sc, Co, Cu, Ca, Sr, Ag, Au, Sn, Bi, At, Rh, Er, Pm, Sm, Ho, Lu, Dy, Gd, Eu, Mn, Pb and Tl, and optionally the radioactive isotopes thereof.

A particular complex of the invention is a complex of a metal element chosen from Pb and Er, with a peranhydro-cyclodextrin derivative, as defined above, with at least one of the values R¹ being a radical chosen from peptides and biopolymers.

These complexes, because of the presence of radicals derived from biomolecules, are capable of being transported to biological targets. Depending on the metal element complexed, they can therefore be used to treat the target with the metal element in question, or can be used in medical imaging.

By virtue of the abovementioned uses, the complexes defined above can be part of the makeup of diagnostic or therapeutic compositions.

As a result, a subject of the invention is thus also diagnostic compositions comprising a complex of a per(3,6-anhydro)cyclodextrin derivative and of a metal element as defined above, and a pharmaceutically acceptable carrier.

In particular, for this type of composition, the metal element forming a complex with a peranhydrocyclodextrin derivative according to the invention is preferably a metal element chosen from metal elements that emit γ-radiation or β⁺-radiation, it being possible for these elements to be chosen from Tc, In, Ga, Co, Cu, Sm and Ga.

It is understood that these elements can exist in several isotope forms.

A subject of the invention is also therapeutic compositions comprising a complex of a per(3,6-anhydro)cyclodextrin derivative and of a metal element as defined above, or a per(3,6-anhydro)cyclodextrin derivative as defined above and a pharmaceutically acceptable carrier.

When such compositions comprise a peranhydrocyclo-dextrin derivative in accordance with the invention, they can be used in particular in the field of decontamination of a target organ or of a biological fluid, because of the ability of the cyclodextrins according to the invention to be able to form complexes with metals.

When such compositions comprise a complex of a metal element, according to the present invention, they are used to transport said complex to a biological target, so that the complexed metal element can exert its therapeutic action on this target.

In particular, such metal elements can be chosen from metal elements that emit β⁻-radiation and α-radiation, it being possible for these elements to be chosen from Sc, Cu, Sr, Y, Rh, Ag, Sn, Pm, Sm, Ho, Lu, Re, Er, Dy, At, Bi, Au, and alkaline earth metals such as Ca.

A subject of the invention is also a method for decontaminating a biological medium (biological target or fluid) with respect to at least one metal element in vitro, said method comprising a step consisting in bringing said biological medium into contact with a per(3,6-anhydro)cyclodextrin derivative as defined above, so as to attach said elements in the form of a complex with a per(3,6-anhydro)cyclodextrin derivative according to the invention.

The metal elements that can be attached or separated by means of the method of the invention may be of various types.

Thus, these metal elements can be chosen from Tc, Y, In, Ga, Re, Sc, Co, Cu, Ca, Sr, Ag, Au, Sn, Bi, At, Rh, Er, Pm, Sm, Ho, Lu, Dy, Gd, Eu, Mn, Pb and Tl, and optionally the radioactive isotopes thereof.

In particular, toxic elements such as lead pollute the environment and may be toxic both in animals and in humans. It is therefore necessary to separate and to eliminate these elements from the environment. Furthermore, products that would make it possible to ensure the decontamination of living beings without affecting their nervous system and other organs would constitute a great advantage.

According to the invention, it has been found that per(3,6-anhydro)cyclodextrin derivatives corresponding to formulae (I) and (II) given above exhibit a particularly advantageous ability to attach the metal ions mentioned above.

In particular, advantageous derivatives are per(3,6-anhydro)cyclodextrin derivatives corresponding to the formula (I) in which an R¹ represents a radical chosen from peptides or biopolymers, the other values R¹ representing —OH, R² linked to the R¹ that represents a peptide radical or biopolymer represents a spacer group of formula —OCH₂CO—, the values R² linked to the values R¹ that represent —OH are single bonds, and n is equal to 6, and more particularly that with a peptide radical R¹ of formula —NH—CH(CH₃)—CO—NH—CHBz—CO—OCH₃ with Bz representing a benzyl group, and that with an R¹ corresponding to a biopolymer of the poly-L-lysine type.

According to the invention, it has been found that derivatives as defined above have a particularly advantageous ability to attach the metal elements mentioned above.

In particular, a crosslinked per(3,6-anhydro)cyclo-dextrin derivative for which all the values R¹ are poly-L-lysines and the values R² correspond to the spacer group of formula —O—CH₂—CO—, and n is equal to 6, at least some of the abovementioned values R¹ being crosslinked by the action of glutaraldehyde in the presence of sodium borohydride, is particularly advantageous for decontamination.

These abovementioned particular derivatives exhibit in particular a high specificity for attaching lead and erbium.

They may therefore find applications, in particular when the ion to be attached is lead, in the field of human decontamination targeted to certain organs.

Such derivatives are particularly advantageous for the following reasons:
they are biocompatible and can therefore be used in vivo;
they can be used for in vitro decontamination of physiological liquids, in particular when they are crosslinked, as defined above; these derivatives, that are generally insoluble in physiological liquids, can be adsorbed onto filtration membranes, said physiological liquid to be decontaminated passing through said membrane.

Other advantages and characteristics of the invention will emerge more clearly on reading the examples which follow, given by way of nonlimiting illustration with reference to the attached drawings.

### BRIEF DESCRIPTION OF THE DRAWINGS

FIG. 1 represents the NMR spectrum in deuterated water of the crude product (i.e. unpurified) prepared in Example 1, with a part B representing this spectrum in the zone from 1 to 7.5 ppm and a part A being an enlarged zone of the zone from 1.2 to 1.6 ppm.

FIG. 2 represents the NMR spectrum in dimethyl sulphoxide of the crude product (i.e. unpurified) prepared in Example 1, with a part A representing this spectrum in the zone from 1 to 9 ppm, a part B being an enlarged zone of this same spectrum between 1 and 1.5 ppm and a part C being an enlarged zone of this same spectrum between 7.5 and 9 ppm.

FIG. 3 represents an NMR spectrum in deuterated water, with a part A representing the spectrum of poly-L-lysine and a part B representing the spectrum of the product prepared in Example 2.

FIG. 4 represents a diagrammatic representation of the inverse of the retention fractions (1/Rf) for Pb²⁺ and Er³⁺, respectively for mono-2-O-carboxymethyl-per(3,6-anhydro) α-cyclodextrin (1), the product prepared in Example 1 (2) and the product prepared in Example 2 (3).

### DETAILED DISCLOSURE OF PARTICULAR EMBODIMENTS

### EXAMPLE 1

This example corresponds to the preparation of [(mono-2-O-methylamido)-per(3,6-anhydro)-α-cyclodextrin]-L-Ala-L-Phe OMe ester.

This compound corresponds to formula (I), in which an R¹ corresponds to the dipeptide L-alanyl-L-phenylalanine-OMe, i.e. corresponds to the formula —NH—CH(CH₃) —CO—NH—CH(Bz)—COOCH₃, the associated R² being a spacer group of formula —O—CH₂—CO—, the other values R¹ correspond to —OH, the associated values R² representing a single bond, and n is equal to 6.

15 mg (0.017 mmol) of mono-2-O-carboxymethyl-per-(3,6-anhydro) α-cyclodextrin (referred to as base cyclodextrin) that had been freshly lyophilized are weighed out, and 6.6 mg (0.032 mmol, 2 equivalents) of dicyclohexylcarbodiimide (DCCI) recrystallized from ethyl acetate are added thereto. These two products are dissolved in 3 ml of anhydrous dimethylformamide. The mixture is stirred for 10 minutes under a stream of dry nitrogen. 5.1 mg (0.020 mmol) of freshly lyophilized L-alanyl-L-phenylalanine-Omethyl ester are then added and the mixture is left to stir for 48 hours. The solvent is evaporated off under vacuum and the evaporation residue is taken up with distilled water and filtered through a 0.22 μm filter (Millex®-SLGS 025 OS) in order to remove the dicyclohexylurea. The clear solution obtained is lyophilized and characterized by thin layer chromatography and proton nuclear magnetic resonance (500 MHz, 298 K).

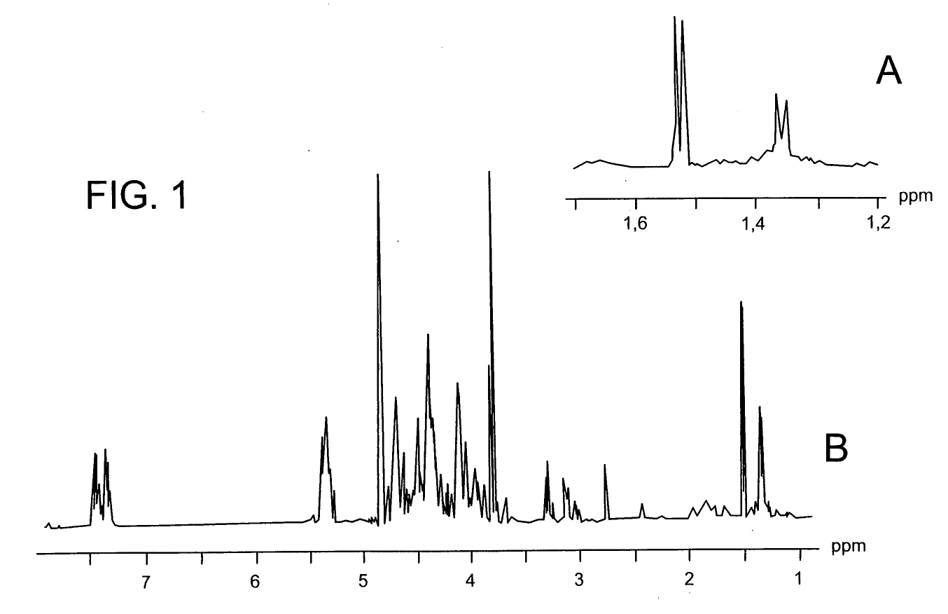

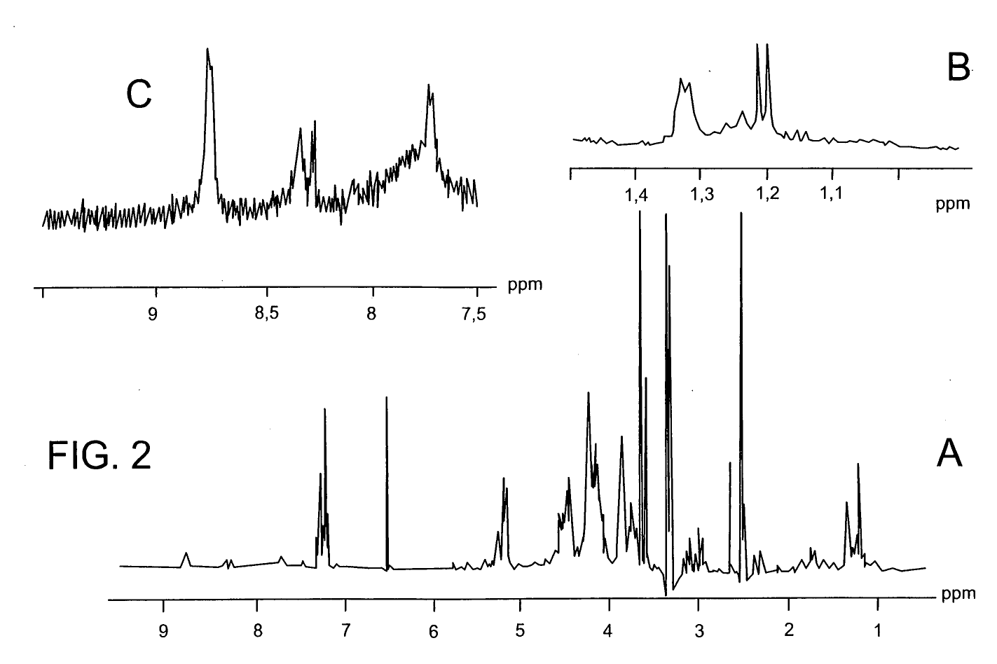

FIGS. 1 and 2 represent, respectively, the spectra of the crude product (i.e. unpurified) in deuterated water and deuterated dimethyl sulphoxide.

The thin layer chromatography (silica gel, eluent: 1/1/1 butanol/DMF/water) shows the presence of a product exhibiting an Rf of 0.48, which is different from that of the free cyclodextrin (Rf=0.37) and of the free dipeptide (Rf=0.77).

The proton spectrum (500 MHz, 298 K), in deuterated water, of the crude compound prepared in this example (i.e. unpurified, or, in other words, still in the presence of the free dipeptide and of base 3,6-cyclo-dextrin) is represented in FIG. 1, in two parts:
a part B representing this spectrum in the zone from 1 to 7.5 ppm; and
a part A being an enlarged zone of the spectrum between 1.2 and 1.6 ppm.

This spectrum shows a doublet at 1.345 ppm corresponding to the β-methyl of the L-alanyl group grafted onto the cyclodextrin and having the same coupling constant (7 Hz) as the doublet of the β-methyl of the L-alanyl group of the free dipeptide. The latter appears at 1.525 ppm.

The proton spectrum (500 MHz, 298 K) in deuterated DMSO, of the crude compound prepared in this example (i.e. unpurified, or, in other words, still in the presence of the free dipeptide and of base 3,6-cyclo-dextrin) is represented in FIG. 2, in three parts:
a part A representing this spectrum in a zone from 1 to 9 ppm;
a part B representing an enlarged zone of this same spectrum between 1 and 1.5 ppm; and
a part C representing an enlarged zone of this same spectrum between 7.5 and 9 ppm. #On this spectrum, two peaks at 8.375 and 7.725 ppm, respectively, appear in the zone where the protons of the peptide bonds emerge. These signals correspond to the signals of the —NH— protons of the two peptide bonds carried by the grafted cyclodextrin and differ from the signal of the —NH— proton of the peptide bond of the free dipeptide, located at 8.785 ppm. These two signals disappear when deuterated water is added, which confirms that they are indeed the —NH— protons of the peptide bonds.

### EXAMPLE 2

This example corresponds to the preparation of [(mono-2-O-methyl(mono-ε-amidopoly-L-Lysine)]-per-(3,6-anhydro)-α-cyclodextrin.

This compound corresponds to formula (I) in which an R¹ corresponds to a poly-L-lysine (PLL), the associated R² being a spacer group —O—CH₂—CO—, said PLL being a commercial PLL with an average molecular mass of 7500 and the other values R¹ correspond to —OH, the associated values R² representing single bonds, and n is equal to 6.

10 mg (0.0108 mmol) of freshly lyophilized mono-2-O-carboxymethyl-per-(3,6-anhydro)cyclomaltohexaose are weighed out, and 8 mg (0.039 mmol) of recrystallized dicyclohexylcarbodiimide (DCC) are added thereto. These two products are dissolved in 3 ml of anhydrous DMF. The mixture is left to stir under a stream of dry nitrogen for 15 minutes, before adding 4 mg (5×10⁻⁴ mmol, i.e. 0.019×10⁻³ —NH₂ functions on average) of PLL. The stream of nitrogen is withdrawn, the round-bottomed reaction flask is stoppered and the solution is left to stir for 48 hours.

The solvent is evaporated off under vacuum and the evaporation residue is taken up with water. The product is then dialyzed against 10⁻³ M HCl for 48 hours using a cellulose ester membrane (Spectra/Por® CE, cutoff threshold 1000), and lyophilized.

The final product is then characterized by thin layer chromatography and by NMR.

On a silica plate and using the following mixture of solvents: butanol/DMF/water (1/1/1), a product is demonstrated for which the Rf is 0.64 and different from the nongrafted cyclodextrin, for which the value is 0.44, and from the free PLL, which does not migrate.

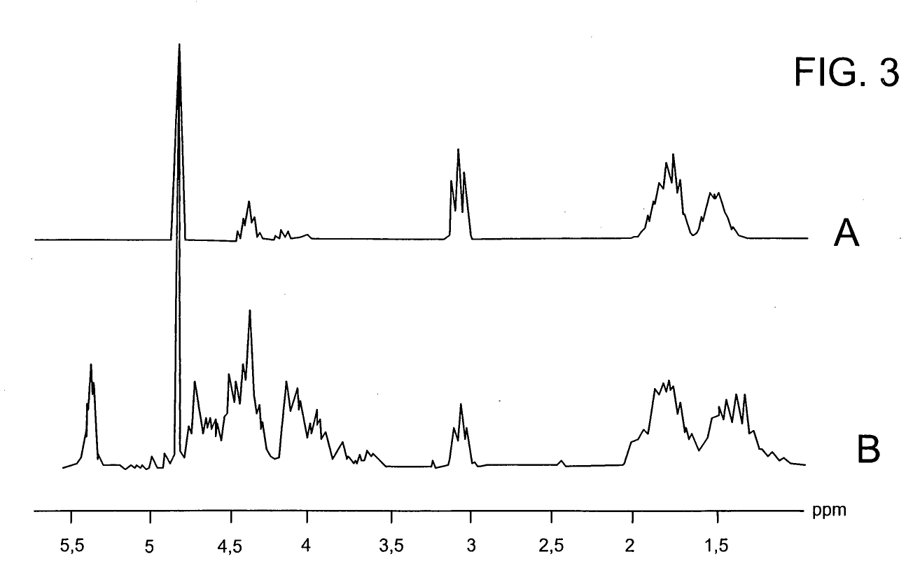

FIG. 3 shows the proton NMR spectra (200 MHz, 298 K, D₂O) of the free PLL (5 mM) (part A) and of the final product of the reaction (Figure B), each one after dialysis under the same conditions as above. The spectrum in part B clearly represents the spectrum of the cyclodextrin coupled to PLL, the free 3,6-cyclo-dextrin having crossed the membrane. A simple test makes it possible to confirm that the free 3,6-cyclodextrin passes out through the membrane and that the PLL is kept inside the membrane. Specifically, if an equimolar mixture of free PLL and of noncoupled mono-2-O-carboxymethyl-per-(3,6-anhydro)cyclomaltohexaose is dialyzed, having taken care to use a Spectra/Por® with a cutoff threshold of 1000, the residue after dialysis contains only the free PLL.

The crude product obtained after lyophilization is subsequently crosslinked by adding two equivalents of glutaraldehyde and two equivalents of NaBH₄ in DMF, per equivalent of PLL-coupled cyclodextrin. After evaporation of the solvent, the product obtained is insoluble in water, which is sufficient to show that there is indeed crosslinking of the PLL.

### EXAMPLE 3

In this example, it is a question of showing the properties of complexation of the compounds prepared in Examples 1 and 2, with respect to cations used in nuclear medicine.

To evaluate the complexation, ion-loaded thin layer chromatography plates which allow rapid evaluation of the complexation of these ions by the species to be evaluated are used. In the present case, plates of Polygram Ionex 25-SA-Na type (Macherey-Nagel, ref: 80613) loaded with various counterions were used. In each test, the compound to be tested is introduced onto the plate, and if the compound complexes the ions, they will be retained on the plate. The plates are then developed four times in water, because of the small amount of migration of the cyclodextrin derivatives, and then the degree of complexation is determined through the value 1/Rf, where Rf represents the ratio: (distance covered by the cyclodextrin derivative/distance covered by the solvent).

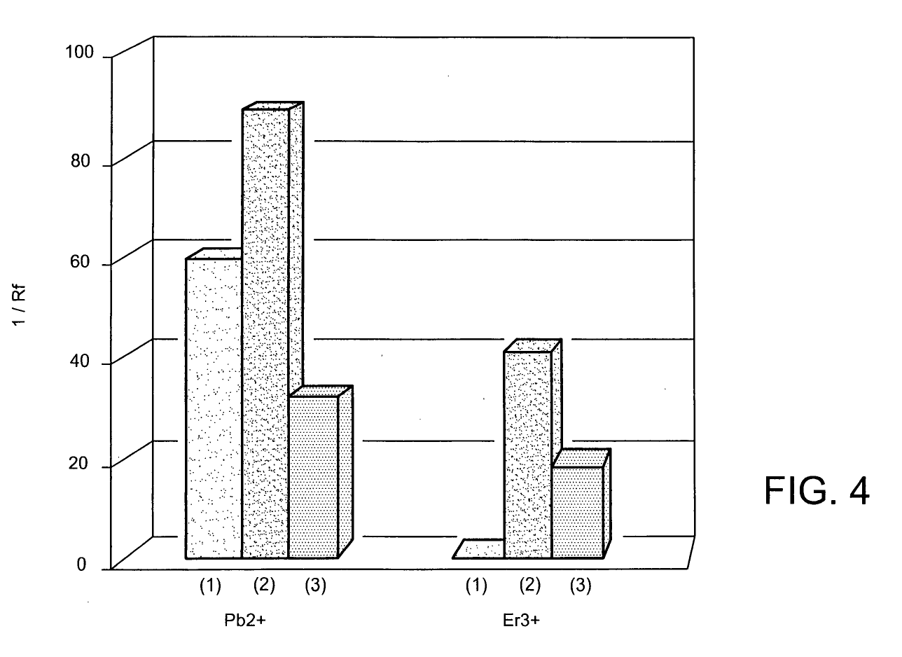

Measurements of degree of complexation of lead (in the form Pb²⁺) and of erbium in the form (Er³⁺) were carried out for the following three compounds:
the base cyclodextrin mono-2-O-carboxyméthyle-per-(3,6-anhydro)cyclomaltohexaose (designated (1) in FIG. 4);
a cyclodextrin in accordance with the invention, prepared in the context of Example 1 (denoted (2) in FIG. 4);
a cyclodextrin in accordance with the invention, prepared in the context of Example 2 (denoted (3) in FIG. 4).

FIG. 4 shows that the lead- and erbium-complexation properties of the compounds prepared in Examples 1 and 2, and in particular of the compound prepared in the context of Example 2, are particularly advantageous.

As regards erbium, FIG. 3 shows in particular that the compounds prepared in Examples 1 and 2 complex erbium, whereas the noncoupled per(3,6-anhydro)cyclodextrin does not complex erbium.

The compounds prepared in the context of the invention can thus be advantageous in human decontamination (in the case of lead contamination) and in nuclear medicine, insofar as erbium is a β⁻-radiation emitter used in radiotherapy, and are particularly advantageous since they are biocompatible due to the presence, in their structure, of radicals of peptide nature or of biopolymer.

### REFERENCES CITED

[1] D. Duchêne “Pharmaceutical application of cyclodextrins” in “Cyclodextrins and their industrial uses”. D. Duchêne Ed., Editions de Santé, Paris, 1987, pages 213-257;
[2] Gadelle A. et Defaye J., Angew. Chem. Int. Ed. Engl., (1991), 30, pages 78-79;
[3] Ashton P. R., Ellwood P., Staton I. and Stoddart J. F., Angew. Chem. Int. ed. Engl., (1991) 30, pages 80-81);
[4] Yamamura H. and Fujita K. Chem. Pharm. Bull., (1991) 39, pages 2505-2508;
[5] Yamamura H., Ezuka T., Kawase Y., Kawai M., Butsugan Y. and Fujita K., J. Chem. Soc., Chem. Com., (1993), pages 636-637;
[6] Yamamura H. Nagaoka H., Kawai M. and Butsugan Y., Tetrahedron Lett. (1995) 36, pages 1093-1094);
[7] FR-A 2 744 124;
[8] FR-A 2 764 525;
[9] FR-A 2 807 044.

1.-24. (canceled)

25. A per(3,6-anhydro)cyclodextrin derivative corresponding to one of the following formulae (I) or (II): 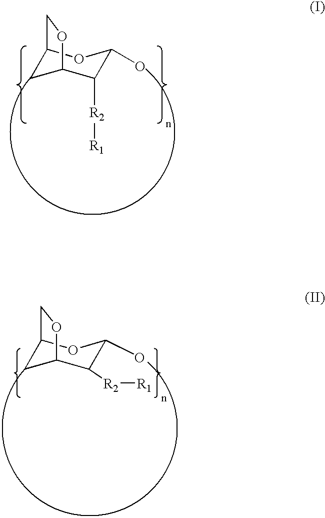
in which:
at least one of the values R¹ represents a radical chosen from peptides, proteins, lipids, oligonucleotides or polynucleotides, oligosaccharides or polysaccharides, and biopolymers;
the other possible values R¹, which may be identical or different, represent a group chosen from OH, OR³, OM, SH, SR³, OCOR³, NH₂, NHR³, NR³R⁴, CONH₂, CONHR³, CONR³R⁴, CN, COOR³, OCH₂COOH, COOH, OSO₂R³, N₃ and R³, in which R³ and R⁴, which may be identical or different, represent a saturated or unsaturated, aliphatic or aromatic, hydrocarbon-based group optionally substituted with halogen atoms, which may contain one or more hetero atoms chosen from O, S and N, and M represents a monovalent cation chosen from alkali metal cations;
the values R², which may be identical or different, represent a single bond or a spacer group; and
n is equal to 6, 7 or 8.

26. A per(3,6-anhydro)cyclodextrin derivative according to claim 25, in which the spacer group is a group chosen from —NH—, —NR—, —NH—(CH₂)ₘ—CO₂—, —NH—(CH₂)ₘ—NH—, —NH—CO₂—, —NH-CO—, —NH-CO—(CH₂)ₘ—COO—, —NH-CO—(CH₂)ₘ—CO—NH—, —NH—CO—(CH₂)ₘ—NH-CO—, —NH—CO—CHR-NH—, —O—, —O—(CH₂)ₘ—, —O—(CH₂)ₘ—O—, —O—(CH₂)ₘ—CO—, —O—(CH₂)ₘ—CO₂—, —O—(CH₂)ₘ—NH—, —O—(CH₂)ₘ—NH—CO—, —O—(CH₂)ₘ—CO—NH—, —O—CO—NH—, —O—CO—NR—, —O—CO—, —O—CO₂—, —S—, —S—(CH₂)ₘ—CO—, —S—(CH₂)ₘ—CO₂—, —S—(CH₂)ₘ—NH—, —S—(CH₂)ₘ—NH—CO—, —S—(CH₂)ₘ—CO—NH—, —S—CO—NH—, —S—CO—, —S—CO—S—, —O—CS—S—, —CS—NR—, —O—CS₂—, —CO—, —CO—NH—, —CO—NR—, —CS—NR—, —CO—O—, —CO—S—, —CO—O—(CH₂)ₘ—CO—O, —CO—S—(CH₂)ₘ—CO—O—, —CO—S—(CH₂)ₘ—CO—S—, —CHOH—NH—, —CHSH—NH—, —CHOH—(CH₂)ₘ—NH—, —CHSH—(CH₂)ₘ—NH— with R having the same definition as R³ and R⁴ of claim 25; m is an integer ranging from 1 to 12.

27. A per(3,6-anhydro)cyclodextrin derivative according to claim 25, in which at least one of the values R¹ represents a radical of peptide nature, the other possible values R¹ representing —OH, the R² linked to R¹ that represents a radical of peptide nature represents a spacer group of formula —OCH₂CO—, the possible values R² linked to the possible values R¹ that represent —OH are single bonds, and n is equal to 6.

28. A per(3,6-anhydro)cyclodextrin derivative according to claim 26, in which at least one of the values R¹ represents a radical of peptide nature, the other possible values R¹ representing —OH, the R² linked to R¹ that represents a radical of peptide nature represents a spacer group of formula —OCH₂CO—, the possible values R² linked to the possible values R¹ that represent —OH are single bonds, and n is equal to 6.

29. A per(3,6-anhydro)cyclodextrin derivative according to claim 27, in which the radical of peptide nature, corresponding to R¹, corresponds to the formula—NH—CH(CH₃)—CO—NH—CHBz—CO—OCH₃, Bz corresponding to a benzyl group.

30. A per(3,6-anhydro)cyclodextrin derivative according to claim 25, in which at least one of the values R¹ represents a biopolymer, the other possible values R¹ representing —OH, R² linked to R¹ that represents a biopolymer represents a spacer group of formula OCH₂CO, the other possible values R² linked to the values R¹ that represent —OH represent single bonds, and n is equal to 6.

31. A per(3,6-anhydro)cyclodextrin derivative according to claim 26, in which at least one of the values R¹ represents a biopolymer, the other possible values R¹ representing —OH, R² linked to R¹ that represents a biopolymer represents a spacer group of formula OCH₂CO, the other possible values R² linked to the values R¹ that represent —OH represent single bonds, and n is equal to 6.

32. A per 3,6-anhydro)cyclodextrin derivative according to claim 30, in which the biopolymer, corresponding to R¹, is a poly-L-lysine.

33. A per(3,6-anhydro)cyclodextrin derivative according to claim 31, in which the biopolymer, corresponding to R¹, is a poly-L-lysine.

34. A per(3,6-anhydro)cyclodextrin derivative according to claim 25, in which said derivative is crosslinked.

35. A derivative according to claim 34, in which the per(3,6-anhydro)cyclodextrin derivative is a derivative for which all the values R¹ are a poly-L-lysine and the values R² are a spacer group of formula —O—CH₂—CO—, n being equal to 6, and at least one of the abovementioned values R¹ being crosslinked by the action of glutaraldehyde in the presence of sodium borohydride.

36. A method for preparing a per(3,6-anhydro)cyclodextrin derivative corresponding to one of the following formulae (I) or (II): 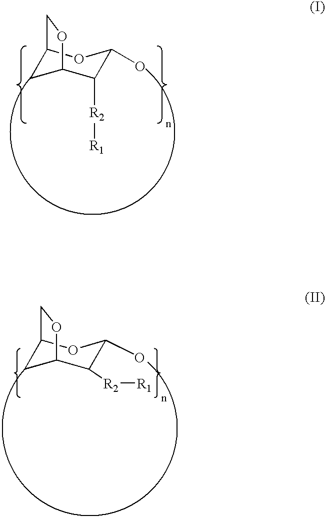
in which at least one of the values R¹ represents a radical derived from a biomolecule chosen from peptides, proteins, lipids, oligonucleotides or polynucleotides, oligosaccharides or polysaccharides, and biopolymers, and the other values R¹, which may be identical or different, represent a group chosen from OH, OR³, OM, SH, SR³, OCOR³, NH₂, NHR³, NR³R⁴, CONH₂, CONHR³, CONR³R⁴, CN, COOR³, OCH₂CO₂H COOH, OSO₂R³, N₃ and R³, in which R³ and R⁴, which may be identical or different, represent a saturated or unsaturated, aliphatic or aromatic, hydrocarbon-based group optionally substituted with halogen atoms, which may contain one or more hetero atoms chosen from O, S and N, M represents a monovalent cation chosen from alkali metal cations, the values R², which may be identical or different, represent a single bond or a spacer group, and n is equal to 6, 7 or 8,
said method comprising the following steps:
1) reacting a peranhydrocyclodextrin of the following formulae (III) or (IV): 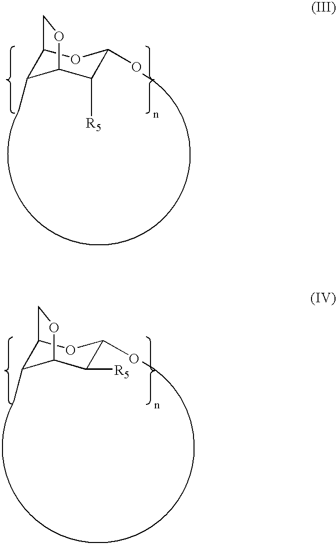
in which the values R⁵, which may be identical or different, represent an OH group or a group that is a precursor of the spacer group R², with a biomolecule chosen from peptides, proteins, lipids, oligonucleotides or polynucleotides, oligosaccharides or polysaccharides, and biopolymers, said biomolecule comprising a reactive group capable of reacting with the group R⁵ as defined above;
2) optionally, reacting the values R⁵ that have not reacted with the abovementioned biomolecule, with one or more suitable reactants so as to convert them to the desired groups R¹.

37. A complex of a metal element with a per(3,6-anhydro)cyclodextrin derivative as defined in claim 25.

38. A complex according to claim 37, in which the metal element forming a complex with the peranhydrocyclodextrin derivative is chosen from Tc, Y, In, Ga, Re, Sc, Co, Cu, Ca, Sr, Ag, Au, Sn, Bi, At, Rh, Er, Pm, Sm, Ho, Lu, Dy, Gd, Eu, Mn, Pb and Tl, and optionally the radioactive isotopes thereof.

39. A complex according to claim 37, in which at least one of the values R¹ is a radical chosen from peptides and biopolymers, and the metal element is Pb or Er.

40. A complex according to claim 38, in which at least one of the values R¹ is a radical chosen from peptides and biopolymers, and the metal element is Pb or Er.

41. A diagnostic composition comprising a complex according to claim 37, and a pharmaceutically acceptable carrier.

42. A composition according to claim 41, wherein the metal element is chosen from metal elements that emit γ-radiation or β⁺-radiation.

43. A composition according to claim 42, wherein the metal element is Tc, In, Co, Cu, Sm, or Ga.

44. A therapeutic composition comprising a complex according to claim 37, or a derivative according to claim 25, and a pharmaceutically acceptable carrier.

45. A therapeutic composition according to claim 44, wherein the metal element is chosen from metal elements that emit β⁻-radiation and α-radiation.

46. A therapeutic composition according to claim 45, wherein the metal element is Sc, Cu, Sr, Y, Rh, Ag, Sn, Pm, Sm, Ho, Lu, Re, Er, Dy, At, Bi, Au or an alkaline earth metal.

47. A method for decontaminating a biological medium with respect to a metal element in vitro, said method comprising a step consisting in bringing said biological medium into contact with a per(3,6-anhydro)cyclodextrin derivative according to claim 25, so as to attach said metal element in the form of a complex with the per(3,6-anhydro)cyclodextrin derivative.

48. A method according to claim 47, wherein the metal is chosen from Tc, Y, In, Ga, Re, Sc, Co, Cu, Ca, Sr, Ag, Au, Sn, Bi, At, Rh, Er, Pm, Sm, Ho, Lu, Dy, Gd, Eu, Mn, Pb and Tl, and optionally the radioactive isotopes thereof.

49. A method according to claim 47, in which the derivative used is a crosslinked derivative according to claim 35.

50. A method according to claim 47, in which the per(3,6-anhydro)cyclodextrin derivative corresponds to formula (I) in which an R¹ represents a radical chosen from peptides and biopolymers, the other values R¹ representing —OH, R² linked to the R¹ that represents a peptide radical or biopolymer represents a spacer group of formula —OCH₂CO—, the values R² linked to the values R¹ that represent —OH represent a single bond, and n is equal to 6.

51. A method according to claim 50, in which the peptide, corresponding to R¹, corresponds to the formula —NH—CH(CH₃)—CO—NH—CHBz—CO—OCH₃, Bz corresponding to a benzyl group.

52. A method according to claim 50, in which the biopolymer, corresponding to R¹, is a poly-L-lysine.

53. A method according to claim 49, in which the metal element to be attached is lead.

54. A method according to claim 49, in which the metal element to be attached is erbium.
